(19) United States
(12) Patent Application Publication
(10) Pub. No.: US 2024/0050485 A1
(43) Pub. Date: Feb. 15, 2024

(54) TREATMENT OF CHRONIC OBSTRUCTIVE PULMONARY DISEASE BY MESENCHYMAL STEM CELL APOPTOTIC BODIES AND COMPOSITIONS THEREOF

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); Feng LIN, Oceanside, CA (US); James VELTMEYER, Oceanside, CA (US)

(21) Appl. No.: 18/449,657

(22) Filed: Aug. 14, 2023

Related U.S. Application Data
(60) Provisional application No. 63/397,503, filed on Aug. 12, 2022.

(51) Int. Cl. A61K 35/28; C12N 5/0775; A61P 11/00

(52) U.S. Cl. CPC A61K 35/28; C12N 5/0663; A61P 11/00

(57) ABSTRACT

Disclosed are means, treatments and compositions of matter useful for treatment of chronic obstructive pulmonary disease (COPD). In one embodiment the invention provides the administration of mesenchymal stem cell apoptotic bodies alone or in combination with “regenerative adjuvants” to prevent and/or reverse reduction in lung function associated with COPD. In other embodiments the invention teaches the utilization of stem cell apoptotic bodies for induction of pulmonary regeneration directly or indirectly.

### CROSS-REFERENCE TO RELATED APPLICATIONS

This application claims priority to U.S. Provisional Application No. 63/397,503, filed Aug. 12, 2022, and titled “Treatment of Chronic Obstructive Pulmonary Disease by Mesenchymal Stem Cell Apoptotic Bodies and Compositions Thereof”, which is hereby incorporated herein in its entirety.

### FIELD OF THE INVENTION

The teachings herein related to methods and compositions for treating chronic obstructive pulmonary disease through the use of apoptotic bodies of regenerative cells, such as mesenchymal stem cells.

### BACKGROUND

There is a need in the art for methods and compositions for treating chronic obstructive pulmonary disease.

### SUMMARY

While the embodiments below are primarily directed to methods, the embodiments can be applied to agents/compositions as well.

Preferred embodiments include methods for preventing or reducing COPD in a patient, the method comprising administering a therapeutically effective amount of apoptotic bodies to a patient.

Preferred methods include embodiments wherein, therapeutically effective amount of apoptotic bodies in the manufacture of a medicament for use in preventing or reducing COPD.

Preferred methods include embodiments wherein the agent for use in preventing or reducing COPD in a patient, wherein the agent comprises a therapeutically effective amount of apoptotic bodies.

Preferred methods include embodiments wherein said apoptotic bodies are collected from mesenchymal stem cells in serum free media.

Preferred methods include embodiments wherein said apoptotic bodies are collected from mesenchymal stem cells in serum containing media.

Preferred embodiments include methods for reducing cognitive decline in a patient with a COPD, wherein said patient has been exposed to an inflammatory trigger, the method comprising administering a therapeutically effective amount of apoptotic bodies derived from one or more cell populations to said patient after exposure of said patient to said inflammatory trigger.

Preferred embodiments include methods of using a therapeutically effective amount of apoptotic bodies derived from one or more cell populations in the manufacture of a medicament for use in reducing COPD a patient with a cognitive disorder, wherein said patient has been exposed to an inflammatory trigger.

Preferred agents for use in reducing COPD in a patient with a cognitive disorder, wherein said patient has been exposed to an inflammatory trigger, and, wherein the agent comprises a therapeutically effective amount of apoptotic bodies derive from one or more cell types.

Preferred embodiments include methods wherein the COPD is associated with an infection.

Preferred embodiments include methods wherein said infection decreases pulmonary stem cell regenerative activity.

Preferred embodiments include methods wherein the cells are pretreated with interferon gamma.

Preferred embodiments include methods further comprising administering a therapeutically effective amount of immature dendritic cells to said patient.

Preferred embodiments include methods wherein the medicament or agent is for administration in combination with a therapeutically effective amount of immature dendritic cells to said patient.

Preferred embodiments include methods wherein the immature dendritic cell is administered, or is for administration, before, after or simultaneously with apoptotic bodies.

Preferred embodiments include methods wherein the immature dendritic cells is co-administered with the apoptotic bodies.

Preferred embodiments include methods wherein the apoptotic bodies are generated by ozone therapy exposure.

Preferred embodiments include methods wherein the apoptotic bodies are administered, or is for administration, together with intravenous ozone gas.

Preferred embodiments include methods wherein the apoptotic bodies are administered, or is for administration to the patient; before commencement of standard COPD therapy; during wherein said apoptotic bodies are collected from mesenchymal stem cells in serum free media.

Preferred embodiments include methods wherein the patient has lung scarring.

Preferred embodiments include methods wherein the patient is a human.

Preferred embodiments include methods wherein the patient is over 18 years of age.

Preferred embodiments include methods wherein the apoptotic bodies are derived from peripheral blood mononuclear cells.

Preferred embodiments include methods wherein the apoptotic bodies are derived from a regenerative cell.

Preferred embodiments include methods said apoptotic cells is generated by exposure of cells to one or more agents capable of causing apoptosis.

Preferred embodiments include methods wherein said agent capable of causing apoptosis is ultraviolet irradiation.

Preferred embodiments include methods wherein said agent capable of causing apoptosis is gamma irradiation.

Preferred embodiments include methods wherein said agent capable of causing apoptosis is X-irradiation.

Preferred embodiments include methods wherein said agent capable of causing apoptosis Is UV irradiation together with psoralen.

Preferred embodiments include methods wherein said agent capable of causing apoptosis Is UV irradiation together with porfimer sodium.

Preferred embodiments include methods wherein said agent capable of causing apoptosis is ozone.

Preferred embodiments include methods wherein said agent capable of causing apoptosis is hydrogen peroxide.

Preferred embodiments include methods when dependent on claim 4, wherein the apoptotic bodies is for administration, to the patient; before commencement of a surgical procedure; during a surgical procedure; or after completion of a surgical procedure, on said patient.

Preferred embodiments include methods wherein the apoptotic bodies are administered, or is for administration to the patient together with xenon gas.

Preferred embodiments include methods wherein apoptotic bodies are administered with gallium.

Preferred embodiments include methods wherein hydrogen gas is added to the procedure.

Preferred embodiments include methods wherein hCG is added to the procedure.

Preferred embodiments include methods wherein Leukine is added to the procedure.

A kit of parts comprising apoptotic bodies and immature dendritic cells for use in preventing or reducing COPD in a patient following a planned inflammatory trigger.

A kit of parts comprising apoptotic bodies and immature dendritic cells for use in reducing cognitive decline in a patient with a cognitive disorder, wherein said patient has been exposed to an inflammatory trigger.

A kit of parts comprising:
Apoptotic bodies;
Dendritic cells; and
instructions for administration of said IL-1 antagoinist and TNFa antagonist to a patient before, during or after a planned inflammatory trigger.

The kit of claim 38 or 40, wherein the planned inflammatory trigger is a surgical procedure or chemotherapy.

A method for preventing or reducing COPD in a patient following a planned inflammatory trigger in said patient, the method comprising administering a therapeutically effective amount of apoptotic bodies to said patient.

Preferred embodiments include methods wherein the planned inflammatory trigger is surgery and the method is for preventing or reducing post-operative cognitive dysfunction (POCD).

Preferred embodiments include methods wherein the planned inflammatory trigger is chemotherapy.

A method for reducing cognitive decline in a patient with a cognitive disorder, wherein said patient has been exposed to an inflammatory trigger, the method comprising administering a therapeutically effective amount of apoptotic bodies to said patient after exposure of said patient to said inflammatory trigger.

Preferred embodiments include methods wherein the cognitive disorder is delirium, Alzheimer's Disease, multiple sclerosis, stroke, Parkinson's Disease, Huntington's Disease, dementia, frontotemporal dementia, vascular dementia, HIV dementia, Post-Traumatic Stress Disorder and/or Rheumatoid Arthritis.

Preferred embodiments include methods wherein the inflammatory trigger is infection, trauma, surgery, vaccination, arthritis, obesity, diabetes, stroke, cardiac arrest, burns, chemotherapy, blast injury, urinary tract infection (UTI), respiratory tract infection (RTI), HIV, poisoning, alcohol or other medication withdrawal, hypoxia, and/or head injury.

Preferred embodiments include methods wherein the POCD is manifested as one or more of memory loss, memory impairment, concentration impairment, delirium, dementia and sickness behaviour.

Preferred embodiments include methods wherein the patient has, or is at risk of developing, delirium, Alzheimer's Disease, multiple sclerosis, stroke, Parkinson's Disease, Huntington's Disease, dementia, frontotemporal dementia, vascular dementia, HIV dementia, Post-Traumatic Stress Disorder and/or Rheumatoid Arthritis.

Preferred embodiments include methods wherein the patient is a human.

Preferred embodiments include methods wherein the patient is less than 20 years of age or over 50 years of age.

Preferred embodiments include methods wherein the apoptotic bodies are derived from umbilical cord mesenchymal stem cells and immature dendritic cells are generated from peripheral blood.

Preferred embodiments include methods wherein the apoptotic bodies are generated by treatment of mesenchymal stem cells with ozone.

Preferred embodiments include methods wherein the ozone gas is administered at 5 micrograms of ozone per milliliter of medical grade oxygen.

Preferred embodiments include methods therein the ozone gas is administered at 1 micrograms of ozone per milliliter of medical grade oxygen.

Preferred embodiments include methods wherein the apoptotic bodies are administered to the patient; before commencement of a surgical procedure; during a surgical procedure; or after completion of a surgical procedure, on said patient.

Preferred embodiments include methods wherein the surgical procedure is a cardiothoracic, an orthopaedic, a neurological, a vascular, a plastic & reconstructive, a gynaecological, an obstetric, a urological, a general, a head & neck, an ear, nose & throat (ENT), a paediatric, a dental, a maxillofacial, an ophthalmic, a pain management, a trauma, or a minor surgical procedure.

Preferred embodiments include methods wherein the general surgical procedure is a colorectal, a hepatobiliary, or an upper gastro-intestinal surgical procedure.

Preferred embodiments include methods wherein the minor surgical procedure is a catheterisation, a minor skin procedure, a minor orthopedic procedure, a nerve block, an endoscopy, a transoesophageal echocardiogram or another minor procedure.

Preferred embodiments include methods wherein the surgical procedure is carried out under general anaesthesia, regional anaesthesia, local anaesthesia, sedation or a combination thereof.

Preferred embodiments include methods wherein said apoptotic bodies derived from a regenerative cell.

Preferred embodiments include methods wherein said apoptotic bodies are administered together with a Sertoli cell and/or apoptotic bodies derived from a Sertoli cell.

Preferred embodiments include methods wherein said regenerative cells are activated prior to induction to undergo apoptosis.

Preferred embodiments include methods wherein said regenerative cells are activated by contact with allogeneic T cells.

Preferred embodiments include methods wherein said regenerative cells are activated by culture with an inflammatory cytokine.

Preferred embodiments include methods wherein said regenerative cells are activated by culture with an activator of NF-kappa B.

Preferred embodiments include methods wherein said regenerative cells are activated by culture with an activator of the JAK-STAT pathway.

Preferred embodiments include methods wherein said regenerative cells are activated by culture with an activator of NF-kappa B.

Preferred embodiments include methods wherein said regenerative cells are activated by culture with an activator of the toll like receptor pathway.

Preferred embodiments include methods wherein said regenerative cells are activated by culture with an activator of the retinoic acid inducible gene.

Preferred embodiments include methods wherein said regenerative cells are activated by culture with an activator of the melanoma differentiation associated gene-5.

Preferred embodiments include methods wherein said regenerative cells are activated by culture with an activator of the nucleotide-binding oligomerization domain-containing protein.

Preferred embodiments include methods wherein said regenerative cells are activated by treatment with interleukin-1.

Preferred embodiments include methods wherein said regenerative cells are activated by treatment with interferon alpha.

Preferred embodiments include methods wherein said regenerative cells are activated by treatment with interferon beta.

Preferred embodiments include methods wherein said regenerative cells are activated by treatment with interferon gamma.

Preferred embodiments include methods wherein said regenerative cells are activated by treatment with interferon omega.

Preferred embodiments include methods wherein said regenerative cells are activated by treatment with Poly IC.

Preferred embodiments include methods wherein said regenerative cells are activated by treatment with lipopolysaccharide.

Preferred embodiments include methods wherein said regenerative cells are activated by treatment with low molecular weight hyaluronic acid.

Preferred embodiments include methods wherein said regenerative cells are activated by treatment with CpG motifs.

Preferred embodiments include methods wherein said regenerative cells are activated by treatment with neutrophil extracellular traps.

Preferred embodiments include methods wherein said regenerative cell is a stem cell.

Preferred embodiments include methods wherein said stem cell is a hematopoietic stem cell.

Preferred embodiments include methods wherein said hematopoietic stem cell is capable of generating leukocytic, lymphocytic, thrombocytic and erythrocytic cells when transplanted into an immunodeficient animal.

Preferred embodiments include methods wherein said hematopoietic stem cell is non-adherent to plastic.

Preferred embodiments include methods wherein said hematopoietic stem cell is adherent to plastic.

Preferred embodiments include methods wherein said hematopoietic stem cell is exposed to hyperthermia.

Preferred embodiments include methods wherein said hematopoietic stem cell expresses interleukin-3 receptor.

Preferred embodiments include methods wherein said hematopoietic stem cell expresses interleukin-1 receptor.

Preferred embodiments include methods wherein said hematopoietic stem cell expresses c-met.

Preferred embodiments include methods wherein said hematopoietic stem cell expresses mpl.

Preferred embodiments include methods wherein said hematopoietic stem cell expresses interleukin-11 receptor.

Preferred embodiments include methods wherein said hematopoietic stem cell expresses G-CSF receptor.

Preferred embodiments include methods wherein said hematopoietic stem cell expresses GM-CS F receptor.

Preferred embodiments include methods wherein said hematopoietic stem cell expresses M-CSF receptor.

Preferred embodiments include methods wherein said hematopoietic stem cell expresses VEGF-receptor.

Preferred embodiments include methods wherein said hematopoietic stem cell expresses c-kit.

Preferred embodiments include methods wherein said hematopoietic stem cell expresses CD33.

Preferred embodiments include methods wherein said hematopoietic stem cell expresses CD133.

Preferred embodiments include methods wherein said hematopoietic stem cell expresses CD34.

Preferred embodiments include methods wherein said hematopoietic stem cell expresses Fas ligand.

Preferred embodiments include methods wherein said hematopoietic stem cell does not express lineage markers.

Preferred embodiments include methods wherein said hematopoietic stem cell does not express CD14.

Preferred embodiments include methods wherein said hematopoietic stem cell does not express CD16.

Preferred embodiments include methods wherein said hematopoietic stem cell does not express CD3.

Preferred embodiments include methods wherein said hematopoietic stem cell does not express CD56.

Preferred embodiments include methods wherein said hematopoietic stem cell does not express CD38.

Preferred embodiments include methods wherein said hematopoietic stem cell does not express CD30.

Preferred embodiments include methods wherein said regenerative cell is a mesenchymal stem cell.

Preferred embodiments include methods wherein said mesenchymal stem cells are naturally occurring mesenchymal stem cells.

Preferred embodiments include methods wherein said mesenchymal stem cells are generated in vitro.

Preferred embodiments include methods wherein said naturally occurring mesenchymal stem cells are tissue derived.

Preferred embodiments include methods wherein said naturally occurring mesenchymal stem cells are derived from a bodily fluid.

Preferred embodiments include methods wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred embodiments include methods wherein said tissue derived mesenchymal stem cells are isolated from tissues containing cells selected from a group of cells comprising of: endothelial cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue stems, corneal keratocytes, tendon stems, bone marrow reticular tissue stems, nonepithelial stems, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred embodiments include methods wherein said mesenchymal stem cells are plastic adherent.

Preferred embodiments include methods wherein said mesenchymal stem cells express a marker selected from a group comprising of: a) CD73; b) CD90; and c) CD105.

Preferred embodiments include methods wherein said mesenchymal stem cells lack expression of a marker selected from a group comprising of: a) CD14; b) CD45; and c) CD34.

Preferred embodiments include methods wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred embodiments include methods wherein said mesenchymal stem cells from umbilical cord tissue do not express markers selected from a group comprising of: a) CD117; b) CD31; c) CD34; and CD45;

Preferred embodiments include methods wherein said mesenchymal stem cells from umbilical cord tissue express, relative to a human stem, increased levels of interleukin 8 and reticulon 1

Preferred embodiments include methods wherein said mesenchymal stem cells from umbilical cord tissue have the potential to differentiate into cells of at least a skeletal muscle, vascular smooth muscle, pericyte or vascular endothelium phenotype.

Preferred embodiments include methods wherein said mesenchymal stem cells from umbilical cord tissue express markers selected from a group comprising of: a) CD10; b) CD13; c) CD44; d) CD73; and e) CD90.

Preferred embodiments include methods wherein said umbilical cord tissue mesenchymal stem cell is an isolated umbilical cord tissue cell isolated from umbilical cord tissue substantially free of blood that is capable of self-renewal and expansion in culture,

Preferred embodiments include methods wherein said umbilical cord tissue mesenchymal stem cells has the potential to differentiate into cells of other phenotypes.

Preferred embodiments include methods wherein said other phenotypes comprise: a) osteocytic; b) adipogenic; and c) chondrogenic differentiation.

Preferred embodiments include methods wherein said cord tissue derived mesenchymal stem cells can undergo at least 20 doublings in culture.

Preferred embodiments include methods wherein said cord tissue derived mesenchymal stem cell maintains a normal karyotype upon passaging

Preferred embodiments include methods wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred embodiments include methods wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred embodiments include methods wherein said umbilical cord tissue-derived cell secretes factors selected from a group comprising of: a) MCP-1; b) MIPlbeta; c) IL-6; d) IL-8; e) GCP-2; f) HGF; g) KGF; h) FGF; i) HB-EGF; j) BDNF; k) TPO; l) RANTES; and m) TIMP1

Preferred embodiments include methods wherein said umbilical cord tissue derived cells express markers selected from a group comprising of: a) TRA1-60; b) TRA1-81; c) SSEA3; d) SSEA4; and e) NANOG.

Preferred embodiments include methods wherein said umbilical cord tissue-derived cells are positive for alkaline phosphatase staining.

Preferred embodiments include methods wherein said cognitive decline is associated with COVID-19.

Preferred embodiments include methods wherein said cognitive decline is associated with chronic traumatic encephalopathy.

Preferred embodiments include methods wherein said cognitive decline is associated with brain injury.

### DETAILED DESCRIPTION OF THE INVENTION

The invention teaches the use of apoptotic bodies, in particular embodiments apoptotic bodies from mesenchymal stem cells, as a treatment for COPD. In one particular embodiment, the invention teaches the utilization of apoptotic bodies as a means of treatment of COPD

COPD is a consistently progressive, ultimately fatal disease for which no treatment exists capable of either reversing or even interrupting its course. It afflicts more than 5% of the population in many countries, and it accordingly represents the third most frequent cause of death in the U.S., where it accounts for more than 600 billion in health care costs, morbidity, and mortality. COPD possesses several features making it ideal for stem cell-based interventions: a) the quality of life and lack of progress demands the ethical exploration of novel approaches. For example, bone marrow stem cells have been used in over a thousand cardiac patients with some indication of efficacy [1, 2].

Mesenchymal Stem Cells (MSCs) are potent immunomodulatory cells that recognize sites of injury, limit effector T cell reactions, and stimulate regulatory cell populations (i.e., T-regs) via growth factors, cytokines, and other mediators. Simultaneously, MSCs also stimulate local tissue regeneration via paracrine effects inducing angiogenic, anti-fibrotic and remodeling responses [3]. Consequently, MSCs-based therapy represents a viable treatment option for autoimmune conditions and other inflammatory disorders [4-9], yielding beneficial effects in models of autoimmune Type 1 Diabetes [10-16], Systemic Lupus Erythematosus, Autoimmune Encephalomyelitis [17], Multiple Sclerosis [18, 19], cardiac insufficiency [20, 21], and organ transplantation [22]. MSCs have been reported to inhibit inflammation and fibrosis in the lungs [23-26], have shown safety in clinical trials for ARDS [27-30], and have been recently suggested as useful to treat patients with severe COVID-19 based on their effects preventing or attenuating the immunopathogenic cytokine storm [31-34].

Unfortunately, evaluation of stem cell therapy in COPD has lagged behind other areas of regenerative investigation; b) the underlying cause of COPD appears to be inflammatory and/or immunologically mediated. The destruction of alveolar tissue is associated with T cell reactivity [35, 36], pathological pulmonary macrophage activation [37], and auto-antibody production [38]. Mesenchymal stem cells have been demonstrated to potently suppress autoreactive T cells [39, 40], inhibit macrophage activation [41], and autoantibody responses [12]. Additionally, mesenchymal stem cells can be purified in high concentrations from adipose stromal vascular tissue together with high concentrations of T regulatory cells [42], which in animal models are approximately 100 more potent than peripheral T cells at secreting cytokines therapeutic for COPD such as IL-10 [43, 44]. Additionally, use of adipose derived cells has yielded promising clinical results in autoimmune conditions such as multiple sclerosis [42]; and c) Pulmonary stem cells capable of regenerating damaged parenchymal tissue have been reported [45]. Administration of mesenchymal stem cells into neonatal oxygen-damaged lungs, which results in COPD-like alveoli dysplasia, has been demonstrated to yield improvements in two recent publications [46, 47].

Based on the above rationale for stem cell-based COPD treatments, we are proposing a 10 patient Phase I safety trial to assess ability of JadiCell, a type umbilical cord derived stem cells to improve objective and quality of life parameters in patients with moderate to severe COPD.

MSCs can be derived in large number from the Umbilical Cord (UC). JadiCells are a type of UC-MSCs, which can be utilized in the allogeneic setting and have demonstrated safety and efficacy in clinical trials for a number of disease conditions including inflammatory and immune-based diseases. UC-MSCs have been shown to inhibit inflammation and fibrosis in the lungs.

JadiCell UC-MSCs have been utilized to treat patients with severe COVID-19 and have yielded promising results, preventing or attenuating the cytokine storm. JadiCells have been recently introduced intravenously in patients with a neurodegenerative disorder, and have been approved for testing in patients with Type 1 Diabetes (T1D). We hypothesize that JadiCells will exert beneficial therapeutic effects in COPD.

This disclosure relates to apoptotic bodies. The disclosure particularly relates to a composition comprising the apoptotic bodies. The disclosure further relates to preparation of apoptotic bodies from stem cells. Preferably, the apoptotic bodies are prepared from multipotent or pluripotent stem cells, and not from totipotent stem cells. More preferably, the apoptotic bodies are prepared from multipotent stem cells, and not from pluripotent or totipotent stem cells. The disclosure also relates to medical treatments comprising the use of the composition comprising the apoptotic bodies This disclosure relates to a composition. This composition may be suitable for a treatment of a mammal. This composition may comprise an apoptotic body. The apoptotic body may comprise a apoptotic stem cell. In this disclosure, the apoptotic stem cell may comprise any apoptotic stem cell. For example, the apoptotic stem cell may comprise an apoptotic mesenchymal stem cell, an apoptotic embryonic stem cell, an apoptotic fetal stem cell, an apoptotic adult stem cell, an apoptotic amniotic stem cell, an apoptotic cord blood stem cell, an apoptotic induced pluripotent stem cell, or a combination thereof. For example, the apoptotic stem cell may comprise an apoptotic mesenchymal stem cell. For example, the apoptotic stem cell may comprise an apoptotic bone marrow-derived mesenchymal stem cell, an apoptotic dental pulp stem cell, an apoptotic stem cell from human exfoliated deciduous teeth, an apoptotic periodontal ligament stem cell, an apoptotic dental follicle stem cell, an apoptotic tooth germ progenitor cell, an apoptotic stem cell from the apical papilla, an apoptotic oral epithelial progenitor/stem cell, an apoptotic gingiva-derived mesenchymal stem cell, an apoptotic periosteum-derived stem cell, an apoptotic salivary gland-derived stem cell, or a combination thereof. For example, the apoptotic stem cell may comprise an apoptotic body derived from cultured mesenchymal stem cell, an apoptotic body derived from an uncultured gingiva-derived mesenchymal stem cell, an apoptotic dental pulp stem cell, an apoptotic bone-marrow-derived stem cell, or a combination thereof. In this disclosure, the apoptotic stem cell may be obtained by the apoptosis of a stem cell, For example, the apoptosis of a stem cell may be induced by a starvation method, an ultra-violet irradiation method, a thermal stress method, a staurosporine method, or a combination thereof.

For example, the apoptotic stem cell may be obtained by incubating a stem cell in a serum-free medium for a time period in the range of 1 hour to 1,000 hours. Or, the apoptotic stem cell may be obtained by incubating a stem cell in a serum-free medium for a time period in the range of 10 hours to 100 hours. In another example, the apoptotic stem cell may be obtained by heating a stem cell at a temperature in the range of 30.degree. C. to 100.degree. C. for a predetermined period of time. Or, the apoptotic stem cell may be obtained by heating a stem cell at a temperature in the range of 30.degree. C. to 100.degree. C. for a time period in the range of 1 minute to 1,000 minutes. Or, the apoptotic stem cell may be obtained by heating a stem cell at a temperature in the range of 30.degree. C. to 100.degree. C. for a time period in the range of 10 minutes to 100 minutes. Or, the apoptotic stem cell may be obtained by heating a stem cell at a temperature in the range of 40.degree. C. to 70.degree. C. for a predetermined time. Or, the apoptotic stem cell may be obtained by heating a stem cell at a temperature in the range of 40.degree. C. to 70.degree. C. for a time period in the range of 1 minute to 1,000 minutes. Or, the apoptotic stem cell may be obtained by heating a stem cell at a temperature in the range of 40.degree. C. to 70.degree. C. for a time period in the range of 10 minute to 100 minutes. Yet, in another example, the apoptotic stem cell may be obtained by treating a stem cell with staurosporine in an amount in the range of 1 nm staurosporine to 10,000 nM staurosporine for a time period in the range 1 hour to 1,000 hours. Or, the apoptotic stem cell may be obtained by treating a stem cell with staurosporine in an amount in the range of 1 nm staurosporine to 10,000 nM staurosporine for a time period in the range 5 hours to 100 hours. Or, the apoptotic stem cell may be obtained by treating a stem cell with staurosporine in an amount in the range of 100 nm staurosporine to 1,000 nM staurosporine for a time period in the range 1 hour to 1,000 hours. Or, the apoptotic stem cell may be obtained by treating a stem cell with staurosporine in an amount in the range of 100 nm staurosporine to 1,000 nM staurosporine for a time period in the range 5 hours to 100 hours. Or, the apoptotic stem cell may be obtained by treating a stem cell in a serum free medium with staurosporine in an amount in the range of 1 nm staurosporine to 10,000 nM staurosporine for a time period in the range of 1 hour to 1,000 hours. Or, the apoptotic stem cell may be obtained by treating a stem cell in a serum free medium with staurosporine in an amount in the range of 1 nm staurosporine to 10,000 nM staurosporine for a time period in the range of 5 hours to 100 hours. Or, the apoptotic stem cell may be obtained by treating a stem cell in a serum free medium with staurosporine in an amount in the range of 100 nm staurosporine to 1,000 nM staurosporine for a time period in the range of 1 hour to 1,000 hours. Or, the apoptotic stem cell may be obtained by treating a stem cell in a serum free medium with staurosporine in an amount in the range of 100 nm staurosporine to 1,000 nM staurosporine for a time period in the range of 5 hours to 100 hours. Yet, in another example, the apoptotic stem cell may be obtained by irradiating a stem cell at a wavelength in the range of 100 nm to 400 nm for a time period in the range of 0.1 minute to 1,000 minutes. Or, the apoptotic stem cell may be obtained by irradiating a stem cell at a wavelength in range of 100 nm to 400 nm for a UV lamp for a time period in the range of 1 minute to 100 minutes. This disclosure also relates to a method for preparation of the composition of this disclosure. This preparation method may, for example, comprise inducing apoptosis of a stem cell and thereby preparing the apoptotic body comprising the apoptotic stem cell.

In this preparation method, the stem cell may comprise any stem cell. For example, the stem cell may comprise an embryonic stem cell, a fetal stem cell, an adult stem cell, an amniotic stem cell, a cord blood stem cell, an induced pluripotent stem cell, or a combination thereof. For example, the stem cell may comprise a mesenchymal stem cell. For example, the stem cell may comprise a bone marrow-derived mesenchymal cell, a dental pulp stem cell, a stem cell from human exfoliated deciduous teeth, a periodontal ligament stem cell, a dental follicle stem cell, a tooth germ progenitor cell, a stem cell from the apical papilla, an oral epithelial progenitor/stem cell, a gingiva-derived mesenchymal stem cell, a periosteum-derived stem cell, a salivary gland-derived stem cell, or a combination thereof. For example, the stem cell may comprise a cultured mesenchymal stem cell, an uncultured gingiva-derived mesenchymal stem cell, a dental pulp stem cell, a bone-marrow-derived stem cell, or a combination thereof.

In some embodiments therapeutic agents may be added to enhance the effect of the apopotoic bodies on COPD, these include: finasteride, dutasteride (e.g., Avodart), turosteride, bexlosteride, izonsteride, epristeride, epigallocatechin, MK-386, azelaic acid, FCE 28260, and SKF 105,111, ketoconazole, fluconazole, spironolactone, flutamide, diazoxide, 17-alpha-hydroxyprogesterone, 11-alpha-hydroxyprogesterone, ketoconazole, RU58841, dutasteride (marketed as Avodart), fluridil, or QLT-7704, an antiandrogen oligonucleotide, a prostaglandin F2.alpha. analogs, prostaglandin analogs, a prostaglandin, bimatoprost (e.g., Latis se, Lumigan), latanoprost (trade name Xalatan), travoprost (trade name Travatan), tafluprost, unoprostone, dinoprost (trade name Prostin F2 Alpha), AS604872, BOL303259X, PF3187207, carboprost (trade name Hemabate), kopexil (for example, the product Keranique™.), CaCl2, botilinum toxin A, adenosine, ketoconazole, DoxoRx, Docetaxel, FK506, GP11046, GP11511, LGD 1331, ICX-TRC, MTS-01, NEOSH101, HYG-102440, HYG-410, HYG-420, HYG-430, HYG-440, spironolactone, CB-03-01, RK-023, Abatacept, Viviscal®, MorrF, ASC-J9, NP-619, AS101, Metron-F-1, PSK 3841, Targretin (e.g. 1% gel), MedinGel, PF3187207, BOL303259X, AS604872. THG11331, PF-277343, PF-3004459, Raptiva, caffeine, coffee, a herb (such as, e.g., saw palmetto, Glycine soja, Panax ginseng, Castanea sativa, Arnica montana, Hedera helix Geranium maculatum), triamcinolone acetonide, a topical irritant (e.g., anthralin) or sensitizer (e.g., squaric acid dibutyl ester [SADBE] or diphenyl cyclopropenone [DPCP]), clomipramine, unsaturated fatty acids (e.g. gamma linolenic acid), a fatty acid derivative, a thickener (such as, e.g. carbomer, glycol distearate, cetearyl alcohol), a hair loss concealer, niacin, nicotinate esters and salts, adenosine, methionine, an androgen receptor inhibitor, a copper peptide, a compound with superoxide dismutation activity, an agent that increases nitric oxide production (e.g. arginine, citrulline, nitroglycerin, amyl nitrite, or sildenafil (Viagra)), a compound that mobilizes bone marrow-derived stem cells (e.g., growth factors such as G-CSF and/or chemical agents such as plerixafor (Mozobil®)), a compound that regulates the differentiation of stem cells into gender-specific specialized human hair follicles (e.g., finasteride, fluconazole, spironolactone, flutamide, diazoxide, 11-alpha-hydroxyprogesterone, ketoconazole, RU58841, dutasteride, fluridil, or QLT-7704, an antiandrogen oligonucleotide, cyoctol, topical progesterone, topical estrogen, cyproterone acetate, ru58841, combination 5.alpha.-reductase inhibitors, oral contraceptive pills), an antiestrogen, an estrogen, or estrogen-like drug, an anti-oxidants (e.g., glutathione, ascorbic acid, tocopherol, uric acid, or polyphenol antioxidants), inhibitors of reactive oxygen species (ROS) generation (e.g., superoxide dismutase inhibitors; stimulators of ROS breakdown, such as selenium; mTOR inhibitors, such as rapamycin: or sirtuins or activators thereof, such as resveratrol, or other SIRT1. SIRT3 activators, or nicotinamide inhibitors), an agent that induces an immune response or causes inflammation (e.g., tetanus toxoid, topical non-specific irritants (anthralin), or sensitizers (squaric acid dibutyl ester [SADBE] and diphenyl cyclopropenone [DPCP]), and an antiapoptotic compound.

In some embodiments of the invention, growth factors are added to enhance viability of the alveolar unit, and/or promote mitosis. Said growth factors are selected from a group comprising of: BLC, Eotaxin-1, Eotaxin-2, G-CSF, GM-CSF, 1-309, ICAM-1, IFN-gamma, IL-1 alpha, IL-1 beta, IL-1 ra, IL-2, IL-4, IL-5, IL-6, IL-6 sR, IL-7, IL-8, IL-10, IL-11, IL-12 p40, IL-12 p70, IL-13, IL-15, IL-16, IL-17, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Ax1, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 R1, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Angiostatin, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-LACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK. Additionally, angiogenic factors may be added in some embodiments of the invention. Said angiogenic factors include but are not limited to activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, collagen receptors .alpha.sub.1.beta.sub.1 and .alpha.sub.2.beta.sub.1, connexins, Cox-2, ECDGF (endothelial cell-derived growth factor), ECG, ECI, EDM, EGF, EMAP, endoglin, endothelins, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin and fibronectin receptor .alpha.5.beta.1, Factor X, HB-EGF, HBNF, HGF, HUAF, heart derived inhibitor of vascular cell proliferation, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOS s), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alphatransferrin, thrombospondin, urokinase, VEGF-A, VEGF-B, VEGF-C, VEGF-D, VEGF-E, VEGF, VEGF.sub.164, VEGI, EG-VEGF.

For the purpose of the invention, bone marrow mononuclear cells may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest”. In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of ischemic conditions, wherein said cells are administered together with oxytocin.

In the methods of the present invention, autologous bone-marrow is isolated from the subject usually under general anesthesia by aspiration from the tibia, femur, ilium or sternum with a syringe, preferably containing 1 mL heparin with an 18-gauge needle. Bone-marrow mononuclear cells are isolated using standard techniques with which one of skill is familiar; such techniques may be modified depending upon the species of the subject from which the cells are isolated. The marrow cells are transferred to a sterile tube and mixed with an appropriate amount of medium, e.g., 10 mL culture medium (Iscove's modified Dulbecco medium IMDM with 10% fetal bovine serum, penicillin G [100 U/mL] and streptomycin [100.mu.g/mL]). The tube is centrifuged to pellet the bone marrow cells, e.g., at 2000 rpm for five minutes and the cell pellet resuspended in medium, e.g., 5 mL culture medium. Low density bone-marrow mononuclear cells are separated from the suspension, e.g., by density gradient centrifugation over Histopaque-1083™ (Sigma), e.g. as described by Yablonka-Reuveni and Nameroff and hereby incorporated by reference. (Histochemistry (19877) 87:27-38). Briefly, the cell suspension is loaded on 20% to 60% gradient, e.g. Histopaque-1083™. (Sigma), Ficoll-Hypaque or Percoll (both available from Pharmacia, Uppsala, Sweden) according to manufacturer's instructions and as described by Yablonka-Reuveni and Nameroff. For example, the cells are centrifuged at 400 g for 20 minutes for Ficoll-Hypaque or at 2000 rpm for 10 minutes for Percoll. Following centrifugation, the top two-thirds of total volume are transferred into a tube, as these layers contain most of the low density bone-marrow mononuclear cells. The cells are centrifuged, e.g. at 2000 rpm for 10 minutes to remove the Histopaque. This is repeated and the cell pellet of bone-marrow mononuclear cells is resuspended in culture medium or buffer, e.g., IMDM, saline, phosphate buffered saline, for transplantation. Preferably, fresh bone-marrow mononuclear cell, isolated as described above, are used for transplantation.

This invention provides a method of treating diseased tissue in a subject which comprises: a) isolating autologous bone-marrow mononuclear cells from the subject; and b) transplanting locally into the diseased tissue an effective amount of the autologous bone-marrow mononuclear cells, thereby treating the diseased tissue in the subject. In a preferred embodiment the diseased tissue is ischemic tissue or tissue in need of repair or regeneration. The invention teaches that augmentation of levels of oxytocin, locally, or systemically in a patient receiving bone marrow mononuclear cell administration results in increasing angiogenesis in diseased tissue in a subject. The administration of oxytocin is provided to a patient treated with a procedure which comprises: a) isolating autologous bone-marrow mononuclear cells from the subject; and b) transplanting locally into the diseased tissue an effective amount of the autologous bone-marrow mononuclear cells, thereby increasing angiogenesis and repair in the diseased tissue in the subject. In a preferred embodiment the tissue is ischemic tissue or tissue in need of repair or regeneration. This invention also provides a method of preventing heart failure in a subject which is treated with oxytocin and further subjected to a procedure comprising: a) isolating autologous bone-marrow mononuclear cells from the subject; and b) transplanting locally into heart tissue an effective amount of the autologous bone-marrow mononuclear cells so as to result in formation of new blood vessels in the heart tissue, to increase angiogenesis and repair in the heart tissue in the subject, thereby preventing heart failure in the subject. In a preferred embodiment the heart tissue is ischemic heart tissue or heart tissue in need of repair or regeneration after injury or surgery. In other preferred embodiments, compromised or occluded coronary blood vessels are treated by the above-described methods resulting in formation of new blood vessels.

The invention provides a method of utilizing oxytocin administration, either locally, systemically, or in delayed release form for the purpose of augmentation of tissue regeneration in a subject which comprises: a) isolating autologous bone-marrow mononuclear cells from the subject; and b) transplanting locally into the tissue an effective amount of the autologous bone-marrow mononuclear cells, resulting in formation of new blood vessels in the tissue, i.e. increasing angiogenesis and repair in diseased tissue in the subject. In a preferred embodiment the tissue is diseased tissue. More preferably, the diseased tissue is ischemic tissue or damaged tissue in need of repair or regeneration.

In some embodiments, the bone-marrow mononuclear cells may also be cultured in any complete medium containing up to 10% serum, e.g., IMDM containing 10% fetal bovine serum and antibiotics, as described above, for up to four weeks before transplantation. The cells may be cultured with growth factors, e.g., vascular endothelial growth factor. The medium is changed about twice a week. The cultured cells are dissociated from the culture dishes with 0.05% trypsin (Gibco BRL, Grand Island, N.Y.), neutralized with culture medium and collected by centrifugation, for example, at 2000 rpm for five minutes at room temperature. The cells are resuspended in IMDM at a concentration of .apprxeq.1.times.10.sup.5 cells to about 1.times.10.sup.10 cells, preferably about 1.times.10.sup.7 cells to about 1.times.10.sup.8 cells in 50.mu.L for transplantation.

### REFERENCES

ADDIN EN.REFLIST 1. Abdel-Latif, A., et al., Adult bone marrow-derived cells for cardiac repair: a systematic review and meta-analysis. Arch Intern Med, 2007. 167(10): p. 989-97.
2. Martin-Rendon, E., et al., Autologous bone marrow stem cells to treat acute myocardial infarction: a systematic review. Eur Heart J, 2008. 29(15): p. 1807-18.
3. Caplan, A. I. and D. Correa, The MSC: an injury drugstore. Cell Stem Cell, 2011. 9(1): p. 11-5.
4. Ge, W., et al., Regulatory T-cell generation and kidney allograft tolerance induced by mesenchymal stem cells associated with indoleamine 2,3-dioxygenase expression. Transplantation, 2010. 90(12): p. 1312-20.
5. Bartholomew, A., et al., Mesenchymal stem cells suppress lymphocyte proliferation in vitro and prolong skin graft survival in vivo. Experimental hematology, 2002. 30(1): p. 42-8.
6. Casiraghi, F., et al., Pretransplant infusion of mesenchymal stem cells prolongs the survival of a semiallogeneic heart transplant through the generation of regulatory T cells. Journal of immunology, 2008. 181(6): p. 3933-46.
7. Ge, W., et al., Infusion of mesenchymal stem cells and rapamycin synergize to attenuate alloimmune responses and promote cardiac allograft tolerance. American journal of transplantation: official journal of the American Society of Transplantation and the American Society of Transplant Surgeons, 2009. 9(8): p. 1760-72.
8. Uccelli, A., A. Laroni, and M. S. Freedman, Mesenchymal stem cells for the treatment of multiple sclerosis and other neurological diseases. Lancet neurology, 2011. 10(7): p. 649-56.
9. Ge, W., et al., Infusion of mesenchymal stem cells and rapamycin synergize to attenuate alloimmune responses and promote cardiac allograft tolerance. Am J Transplant, 2009. 9(8): p. 1760-72.
10. Perez-Simon, J. A., et al., Mesenchymal stem cells expanded in vitro with human serum for the treatment of acute and chronic graft-versus-host disease: results of a phase I/II clinical trial. Haematologica, 2011. 96(7): p. 1072-6.
11. Garcia-Olmo, D., et al., A phase I clinical trial of the treatment of Crohn's fistula by adipose mesenchymal stem cell transplantation. Dis Colon Rectum, 2005. 48(7): p. 1416-23.
12. Sun, L., et al., Mesenchymal stem cell transplantation reverses multiorgan dysfunction in systemic lupus erythematosus mice and humans. Stem Cells, 2009. 27(6): p. 1421-32.
13. Dhere, T., et al., The safety of autologous and metabolically fit bone marrow mesenchymal stromal cells in medically refractory Crohn's disease-a phase 1 trial with three doses. Aliment Pharmacol Ther, 2016. 44(5): p. 471-81.
14. Carlsson, P. O., et al., Preserved beta-cell function in type I diabetes by mesenchymal stromal cells. Diabetes, 2015. 64(2): p. 587-92.
15. Cai, J., et al., Umbilical Cord Mesenchymal Stromal Cell With Autologous Bone Marrow Cell Transplantation in Established Type 1 Diabetes: A Pilot Randomized Controlled Open-Label Clinical Study to Assess Safety and Impact on Insulin Secretion. Diabetes Care, 2016. 39(1): p. 149-57.
16. Hu, J., et al., Long term effects of the implantation of Wharton's jelly-derived mesenchymal stem cells from the umbilical cord for newly-onset type 1 diabetes mellitus. Endocr J, 2013. 60(3): p. 347-57.
17. Morando, S., et al., The therapeutic effect of mesenchymal stem cell transplantation in experimental autoimmune encephalomyelitis is mediated by peripheral and central mechanisms. Stem cell research & therapy, 2012. 3(1): p. 3.
18. Hedayatpour, A., et al., Promotion of remyelination by adipose mesenchymal stem cell transplantation in a cuprizone model of multiple sclerosis. Cell journal, 2013. 15(2): p. 142-51.
19. Al Jumah, M. A. and M. H. Abumaree, The immunomodulatory and neuroprotective effects of mesenchymal stem cells (MSCs) in experimental autoimmune encephalomyelitis (EAE): a model of multiple sclerosis (MS). Int J Mol Sci, 2012. 13(7): p. 9298-331.
20. Bartolucci, J., et al., Safety and Efficacy of the Intravenous Infusion of Umbilical Cord Mesenchymal Stem Cells in Patients With Heart Failure: A Phase 1/2 Randomized Controlled Trial (RIMECARD Trial [Randomized Clinical Trial of Intravenous Infusion Umbilical Cord Mesenchymal Stem Cells on Cardiopathy]). Circ Res, 2017. 121(10): p. 1192-1204.
21. Tuma, J., et al., RESCUE-HF Trial: Retrograde Delivery of Allogeneic Umbilical Cord Lining Subepithelial Cells in Patients With Heart Failure. Cell Transplant, 2016. 25(9): p. 1713-1721.
22. Franquesa, M., et al., Mesenchymal Stem Cells in Solid Organ Transplantation (MiSOT) Fourth Meeting: lessons learned from first clinical trials. Transplantation, 2013. 96(3): p. 234-8.
23. Moroncini, G., et al., Mesenchymal stromal cells from human umbilical cord prevent the development of lung fibrosis in immunocompetent mice. PLoS One, 2018. 13(6): p. e0196048.
24. Zhang, C., et al., Clinical observation of umbilical cord mesenchymal stem cell treatment of severe idiopathic pulmonary fibrosis: A case report. Exp Ther Med, 2017. 13(5): p. 1922-1926.
25. Moodley, Y., et al., Anti-inflammatory effects of adult stem cells in sustained lung injury: a comparative study. PLoS One, 2013. 8(8): p. e69299.
26. Harrell, C. R., et al., Mesenchymal Stem Cell-Based Therapy of Inflammatory Lung Diseases: Current Understanding and Future Perspectives. Stem Cells Int, 2019. 2019: p. 4236973.
27. Wilson, J. G., et al., Mesenchymal stem (stromal) cells for treatment of ARDS: a phase 1 clinical trial. Lancet Respir Med, 2015. 3(1): p. 24-32.
28. Matthay, M. A., et al., Treatment with allogeneic mesenchymal stromal cells for moderate to severe acute respiratory distress syndrome (START study): a randomised phase 2a safety trial. Lancet Respir Med, 2019. 7(2): p. 154-162.
29. Zhao, R., et al., Serious adverse events of cell therapy for respiratory diseases: a systematic review and meta-analysis. Oncotarget, 2017. 8(18): p. 30511-30523.
30. Laffey, J. G. and M. A. Matthay, Fifty Years of Research in ARDS. Cell-based Therapy for Acute Respiratory Distress Syndrome. Biology and Potential Therapeutic Value. Am J Respir Crit Care Med, 2017. 196(3): p. 266-273.
31. Leng, Z., et al., Transplantation of ACE2-mesenchymal stem cells improves the outcome of patients with COVID-19 pneumonia. ChinaXiv, 2020.
32. Liang, B., et al., Clinical remission of a critically ill COVID-19 patient treated by human umbilical cord mesenchymal stem cells. ChinaXiv, 2020.
33. Zikuan Leng, R. Z., Wei Hou, Yingmei Feng, Yanlei Yang, Qin Han, Guangliang Shan, Fanyan Meng, Dongshu Du, Shihua Wang, Junfen Fan, Wenjing Wang, Luchan Deng, Hongbo Shi, Hongjun Li, Zhongjie Hu, Fengchun Zhang, Jinming Gao, Hongjian Liu, Xiaoxia Li, Yangyang Zhao, Kan Yin, Xijing He, Zhengchao Gao, Yibin Wang, Bo Yang, Ronghua Jin, Ilia Stambler, Lee Wei Lim, Huanxing Su, Alexey Moskalev, Antonio Cano, Sasanka Chakrabarti, Kyung-Jin Min, Georgina Ellison-Hughes, Calogero Caruso, Kunlin Jin, Robert Chunhua Zhao, Transplantation of ACE2<sup>-</sup> Mesenchymal Stem Cells Improves the Outcome of Patients with COVID-19 Pneumonia. Aging and disease, 2020. 11(2): p. 216-228.
34. Peng, Z., Umbilical Cord(UC)-Derived Mesenchymal Stem Cells(MSCs) Treatment for the 2019-novel Coronavirus(nCOV) Pneumonia.
35. Motz, G. T., et al., Persistence of lung CD8 T cell oligoclonal expansions upon smoking cessation in a mouse model of cigarette smoke-induced emphysema. J Immunol, 2008. 181(11): p. 8036-43.
36. Maeno, T., et al., CD8+ T Cells are required for inflammation and destruction in cigarette smoke-induced emphysema in mice. J Immunol, 2007. 178(12): p. 8090-6.
37. Woodruff, P. G., et al., A distinctive alveolar macrophage activation state induced by cigarette smoking. Am J Respir Crit Care Med, 2005. 172(11): p. 1383-92.
38. Stefanska, A. M. and P. T. Walsh, Chronic obstructive pulmonary disease: evidence for an autoimmune component. Cell Mol Immunol, 2009. 6(2): p. 81-6.
39. Gonzalez-Rey, E., et al., Human adipose-derived mesenchymal stem cells reduce inflammatory and T cell responses and induce regulatory T cells in vitro in rheumatoid arthritis. Ann Rheum Dis. 69(1): p. 241-8.
40. Lepelletier, Y., et al., Galectin-1 and Semaphorin-3A are two soluble factors conferring T cell immunosuppression to bone marrow mesenchymal stem cell. Stem Cells Dev, 2009.
41. Tsyb, A. F., et al., In vitro inhibitory effect of mesenchymal stem cells on zymosan-induced production of reactive oxygen species. Bull Exp Biol Med, 2008. 146(1): p. 158-64.
42. Riordan, N. H., et al., Non-expanded adipose stromal vascular fraction cell therapy for multiple sclerosis. J Transl Med, 2009. 7: p. 29.
43. Feuerer, M., et al., Lean, but not obese, fat is enriched for a unique population of regulatory T cells that affect metabolic parameters. Nat Med, 2009. 15(8): p. 930-9.
44. Ogawa, Y., E. A. Duru, and B. T. Ameredes, Role of IL-10 in the resolution of airway inflammation. Curr Mol Med, 2008. 8(5): p. 437-45.
45. Serrano-Mollar, A., et al., Intratracheal transplantation of alveolar type II cells reverses bleomycin-induced lung fibrosis. Am J Respir Crit Care Med, 2007. 176(12): p. 1261-8.
46. Aslam, M., et al., Bone marrow stromal cells attenuate lung injury in a murine model of neonatal chronic lung disease. Am J Respir Crit Care Med, 2009. 180(11): p. 1122-30.
47. van Haaften, T., et al., Airway delivery of mesenchymal stem cells prevents arrested alveolar growth in neonatal lung injury in rats. Am J Respir Crit Care Med, 2009. 180(11): p. 1131-42.

1. A method for preventing or reducing COPD in a patient, the method comprising administering a therapeutically effective amount of apoptotic bodies to a patient.

2. The method of claim 1, wherein said apoptotic bodies are collected from mesenchymal stem cells in serum free media.

3. The method of claim 2, wherein said apoptotic bodies are collected from mesenchymal stem cells in serum containing media.

4. A method for reducing cognitive decline in a patient with a COPD, wherein said patient has been exposed to an inflammatory trigger, the method comprising administering a therapeutically effective amount of apoptotic bodies derived from one or more cell populations to said patient after exposure of said patient to said inflammatory trigger.

5. The method of claim 4, wherein the COPD is associated with an infection.

6. The method of claim 5, wherein said infection decreases pulmonary stem cell regenerative activity.

7. The method of claim 6, wherein the cells are pretreated with interferon gamma.

8. The method of claim 7, further comprising administering a therapeutically effective amount of immature dendritic cells to said patient.

9. The method of claim 4, further comprising administering a therapeutically effective amount of immature dendritic cells to said patient.

10. The method of claim 9, wherein the immature dendritic cell is administered, or is for administration, before, after or simultaneously with apoptotic bodies.

11. The method of claim 10, wherein the immature dendritic cells is co-administered with the apoptotic bodies.

12. The method of claim 11, wherein the apoptotic bodies are generated by ozone therapy exposure.

13. The method of claim 12, wherein the apoptotic bodies are administered, or is for administration, together with intravenous ozone gas.

14. The method of claim 4, wherein the apoptotic bodies are administered, or is for administration to the patient; before commencement of standard COPD therapy; during wherein said apoptotic bodies are collected from mesenchymal stem cells in serum free media.

15. The method of claim 4, wherein the patient has lung scarring.
